(19) United States
(12) Patent Application Publication
(10) Pub. No.: US 2014/0348870 A1
(43) Pub. Date: Nov. 27, 2014

(54) IMMUNOGENIC PLASMODIUM FALCIPARUM ANTIGEN COMPOSITIONS AND USES THEREOF

(71) Applicant: THE REGENTS OF THE UNIVERSITY OF CALIFORNIA, Oakland, CA (US)

(72) Inventors: Philip L. Felgner, Rancho Santa Fe, CA (US); Xiaowu Liang, La Jolla, CA (US); Robert Sauerwein, Nijmegen (NL)

(21) Appl. No.: 14/360,916

(22) PCT Filed: Nov. 30, 2012
(86) PCT No.: PCT/US2012/067245

§ 371 Date: May 27, 2014

Related U.S. Application Data
(60) Provisional application No. 61/565,033, filed on Nov. 30, 2011.

(51) Int. Cl. C07K 14/435

(52) U.S. Cl. CPC C07K 14/43563; A61K 39/015
USPC 424/191.1

(57) ABSTRACT

Contemplated compositions and methods employ selected antigens form Plasmodium falciparum and can be used as a vaccine, therapeutic agent, and/or diagnostic tool. Especially preferred antigens are post-challenge immunity associated antigens that are identified via pre-infection suppressive treatment, controlled sub-symptomatic infection to develop immunity, and comparative proteomic differential analysis.

This application claims priority to copending U.S. provisional application with the Ser. No. 61/565,033, which was filed Nov. 30, 2011.

This invention was made with Government support under Grant Nos. AI066791 and AI075692 awarded by the National Institutes of Health. The Government has certain rights in this invention.

### FIELD OF THE INVENTION

The field of the invention is Plasmodium falciparum antigens, and especially as they relate to their use in prophylactic and/or therapeutic compositions and methods.

### BACKGROUND OF THE INVENTION

Malaria is an infectious disease that is found throughout tropical and subtropical regions of the world. The disease is caused by protists of the genus Plasmodium, which are transmitted by mosquitoes that have ingested a blood meal from an infected individual. While a number of Plasmodium species can infect humans, severe disease is primarily caused by Plasmodium falciparum. Gametocytes from the blood of the infected individual produce sporozoites that localize in the salivary glands of the infected mosquito. Upon biting a new human host, the infected mosquito transfers sporozoites to the new host's blood stream. These sporozoites migrate to the liver, infecting hepatocytes. Here they reproduce asymptomatically to form large numbers of merozoites. Infected hepatocytes rupture to release merozoites into the blood stream, where they infect red blood cells. These merozoites multiply within the red blood cells, where they are periodicaly released to infect more red blood cells. A portion of these merozoites differentiate into gametocytes to perpetuate the organism's life cycle. Symptoms are caused by release of Plasmodium merozoites from the red blood cells, and include fever, chills, and headache. Periodic release of merozoites results in the familiar repeated waves of fever associated with the disease. In severe cases the disease can result in coma or death. While the incidence of malaria is decreasing, the disease continues to have a major impact. In 2009 there were an estimated 225 million cases of malaria worldwide, resulting in an estimated 781,000 deaths (WHO World Malaria Report). Most of these occurred among young children in sub-Saharan Africa.

The impact of malaria can be reduced by treatment and by prevention. The disease can be treated using a variety of anti-malarial drugs including quinine, artmisinin, mefloquine, doxycycline, and chloroquine. Such drugs may be used prophylactically, but long term use entails the risk of negative side effects. In addition the parasite can develop resistance to such drugs (Wellems T E (2002). Science 298 (5591): 124-6). These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling. Unfortunately the expense and side effects of such drugs for the most part restrict their use to short term visitors to regions where malaria is endemic.

Transmission of Plasmodium can be reduced by minimizing the incidence of mosquito bites, either through barriers such as mosquito nets and insect repellents or by control measures such as the use of insecticides or reduction of breeding habitat by draining of standing water. Such measures, however, require continuing effort and expense and may cause inadvertent damage to the environment. Furthermore, aggressive treatment has led to the development of insecticide resistance in the mosquito population.

Due in part to the inherent disadvantages in available control and treatment measures a number of attempts have been made to develop a vaccine that provides sustained and effective protection against malaria. The Plasmodium organism displays a large number of antigenic molecules during its life cycle that may serve as targets for vaccine development, however by living within the host's hepatocytes and erythrocytes the organism can avoid immune surveillance. SPf66 was an early vaccine that utilized synthetic peptides derived from blood stage and sporozoite stage parasites. Unfortunately repeated trials have shown no protective effect (Graves P, Gelband H. (2006) Cochrane Database Syst Rev.; (2):CD005966).

Other vaccine formulations based on blood stage forms of Plasmodium, such as MSP/RESA and MSP2, have reduced parasite load in subsequently infected individuals in some trials or have impacted only specific strains of the parasite (Graves P, Gelband H. (2006) Cochrane Database Syst Rev.; (4):CD006199). The RTS,S vaccine, directed to the pre-erythrocyte stage of the organism, has been found to provide protective effects to a slightly over half of treated patients (Graves P, Gelband H. (2006) Cochrane Database Syst Rev.; (4):CD006198). Efforts to increase this vaccine's effectiveness are ongoing.

Still further known vaccine compositions and related antigens are described in WO 2012/154199, WO 2012/134591, WO 2012/051097, U.S. Pat. App. No. 2010/0183590, U.S. Pat. No. 8,232,255, EP 2 511 367, and EP 1 697 405. Overall, however, to date attempts to produce a vaccine using selected Plasmodium falciparum antigens have demonstrated poor immunogenicity, or have failed to produce a strong antibody responses associated with long term protective effect.

Therefore, even though numerous methods and compositions for diagnosis and prophylaxis of malaia areknown in the art, all or almost all of them sufer from one or more disadvantages. Therefore, there is still a need to provide improved compositions and methods relating to immunogenic Plasmodium falciparum antigens.

### SUMMARY OF THE INVENTION

The inventive subject matter is drawn to compositions and methods for post-challenge immunity associated antigens of Plasmodium falciparum (e.g., LSA-1, CSP, MSP4, and/or SET domain protein, or fragments thereof) in the preparation of vaccines, therapeutics, and diagnostic methods and devices.

In one especially preferred aspect of the inventive subject matter, an antigenic composition includes at least three partially purified post-challenge immunity associated antigens of Plasmodium falciparum, and a carrier associated with the post-challenge immunity associated antigens. While in some aspects the at least three antigens or fragments thereof are LSA-1, CSP, and MSP4, the antigens may also be LSA-1, CSP, and SET domain protein, LSA-1, MSP4, and SET domain protein, or CSP, MSP4, and SET domain protein. Where desired the antigens may also be LSA-1, CSP, MSP4, and SET domain protein, or fragments thereof, all of which may be further accompanied by additional Plasmodium falciparum antigens.

It is further contemplated that the carrier may be a pharmaceutically acceptable carrier, and that the composition is formulated as a vaccine formulation. Alternatively, the carrier may also be a solid phase to which the post-challenge immunity associated antigens are coupled in an individually addressable manner (e.g., to so form part of a disposable diagnostic test device).

In another especially preferred aspect of the inventive subject matter, the inventors contemplate a method of developing a multivalent vaccine formulation that confers persistent immunity against Plasmodium falciparum, and especially preferred methods include a step of identifying a plurality of post-challenge immunity associated antigens of Plasmodium falciparum. In another step, each of the post-challenge immunity associated antigens is at least partially purified, and in a still further step, the at least partially purified post-challenge immunity associated antigens are included into a vaccine formulation comprising a pharmaceutically acceptable carrier and optionally an adjuvant.

It is further generally preferred that the plurality of antigens includes at least one, more typically two, and most typically three antigens selected from the group consisting of LSA-1, CSP, MSP4, and SET domain protein, or fragments thereof. While not limiting to the inventive subject matter, it is also preferred that the step of identifying includes administration of a suppressive drug (e.g., proguanil, chloroquine, mefloquine, doxycycline, etc.) to a mammal prior to a step of infecting the mammal with a dose of sporozoites of Plasmodium falciparum. Most typically, the dose is effective to confer immunity to the mammal without development of symptomatic disease in the mammal.

Therefore, and viewed form a different perspective, the inventors also contemplate a method for assessing the immune competence of an individual to Plasmodium falciparum. In such methods, it is generally preferred to administer a plurality of post-challenge immunity associated antigens of Plasmodium falciparum to the individual (typically naïve to infection with Plasmodium falciparum), to obtain a blood sample from the individual, and to determine quantities of antibodies against each of the post-challenge immunity associated antigens in the blood sample. The determined quantities are then compared against respective threshold values, wherein quantities above the respective threshold values are indicative of immunity to the pathogen.

In further preferred methods, at least one, more typically at least two, and most typically at least three post-challenge immunity associated antigens are selected from the group consisting of LSA-1, CSP, MSP4, and SET domain protein, or fragments thereof. Moreover, it is generally preferred that the respective threshold values are based on a plurality of individuals that have persistent and effective immunity against Plasmodium falciparum.

Various objects, features, aspects and advantages of the present invention will become more apparent from the following detailed description of preferred embodiments of the invention along with the accompanying drawing.

### BRIEF DESCRIPTION OF THE DRAWING

FIGS. 1A and 1B show antibody response to experimental Plasmodium falciparum infection in a volunteer via binding of serum components to an array containing 853 proteins from Plasmodium falciparum before and after being bitten by an uninfected mosquito, respectively. Bright spots indicate that significant amounts of antibody have bound to antigens printed at that location on the array. Locations indicated by circles are background antigens that serve as positive controls.

FIGS. 1C, 1D, and 1E show antibody response to experimental Plasmodium falciparum infection in a volunteer via binding of serum components to an array containing 853 proteins from Plasmodium falciparum at 35, 140, and 400 days, respectively, following a bite from an infected mosquito that resulted in patient parasitemia and clinical disease. Bright spots indicate that significant amounts of antibody have bound to antigens printed at that location on the array. Locations indicated by circles are background antigens that serve as positive controls.

FIGS. 2A and 2B show antibody response to chloroquine prophylaxis followed by Plasmodium falciparum infection (CPS immunization) in a volunteer via binding of serum components to an array containing 853 proteins from Plasmodium falciparum, before and after being bitten by an uninfected mosquito, respectively. Bright spots indicate that significant amounts of antibody have bound to antigens printed at that location on the array. Locations indicated by circles are background antigens that serve as positive controls.

FIGS. 2C, 2D, and 2E show antibody response to chloroquine prophylaxis followed by Plasmodium falciparum infection (CPS immunization) in a volunteer via binding of serum components to an array containing 853 proteins from Plasmodium falciparum. One month after the discontinuation of chloroquine, protection was assessed by homologous challenge with mosquitoes infected with P. falciparum. FIGS. 2C, 2D, and 2E show results from serum at 35, 140, and 400 days, respectively. Bright spots indicate that significant amounts of antibody have bound to antigens printed at that location on the array. Locations indicated by red circles are background antigens that serve as positive controls.

FIG. 3 shows a heatmap that illustrates the differences between the antibody profiles of subjects with CPS immunization and control individuals followed by challenge with mosquitoes infected with Plasmodium falciparum. Arrays were probed with human samples taken 1 day before immunization, 1 day before challenge, and 35 days, 140 days and 400 days post challenge. Shading is related to signal intensity (which is in turn related to the amount of antibody bound to the array at the corresponding location) with dark grey indicating a strong signal, light grey a weak signal and black an intermediate signal.

FIGS. 4A, 4B, and 4C each show the kinetics of the antibody response against different sets of 15 Plasmodium falciparum antigens in non-immunized control subjects following experimental infection. Samples were probed and the results plotted for each antigen prior to exposure to the pathogen (I−1, C−1), 35 days after exposure (C+35), 140 days after exposure (C+145), and 400 days after exposure (C+400).

FIG. 4D shows the kinetics of the antibody response against 6 Plasmodium falciparum antigens in CSP immunized subjects following experimental infection. Samples were probed and the results plotted for each antigen prior to exposure to the pathogen (I−1, C−1), 35 days after exposure (C+35), 140 days after exposure (C+145), and 400 days after exposure (C+400).

FIG. 5A shows average reactivity of control subjects for 853 Plasmodium falciparum antigens at one day pre-challenge and 35 days post challenge. Antigens are plotted on the X-axis and sorted by average signal intensity observed in 35 days post challenge samples. Average array signal intensity for each antigen after pre-immune subtraction is plotted on the Y-axis.

FIG. 5B is a scatterplot showing correlation of data collected from preimmune and one day pre-challenge samples for control subjects.

FIG. 5C shows average reactivity of CSP immunized subjects for 853 Plasmodium falciparum antigens at one day pre-challenge and 35 days post challenge. The 853 antigens were plotted on the X-axis in the same order as in FIG. 5A, and the average signal intensity for each antigen after pre-immune subtraction is plotted on the Y-axis.

FIG. 5D is a scatterplot showing a correlation of data collected from one day before challenge and 35 days post challenge in immunized individuals.

FIG. 5E shows a comparison between the antibody response of immunized individuals 1 day before challenge and naïve individuals 35 days post challenge to 853 Plasmodium falciparum antigens. The 853 antigens were plotted on the X-axis and sorted by the average signal intensity found 35 days post challenge in control subjects, and the average signal intensity for each antigen after pre-immune subtraction is plotted on the Y-axis.

FIG. 5F is a scatterplot showing the correlation of antibody responses in samples collected from one day pre-challenge in immunized individuals and 35 days post challenge in control subjects.

FIG. 6A shows a comparison of antibody responses to selected Plasmodium falciparum antigens associated with unexposed naïve subjects, CPS immunized subjects, experimentally infected subjects, and semiimmune subjects that acquired Plasmodium falciparum naturally. Antibody reactivity against pre-erythrocytic (CS Protein and LSA-1) and blood stage (AMA1, MSP1, MSP2 and LSA3) antigens are compared.

FIG. 6B shows a comparison of antibody responses to a blood stage antigen PfEMP 1 of Plasmodium falciparum associated with unexposed naïve subjects, CPS immunized subjects, experimentally infected subjects, and semiimmune subjects that acquired Plasmodium falciparum naturally.

FIG. 7A shows a comparison of durable LSA-1 antibody responses between CPS immunized subjects and subjects experimentally infected with Plasmodium falciparum. Data points are shown for 1 day prior (C−1) and 35 (C+35), 140 (C+140), and 400 (C+400) days following exposure to the pathogen.

FIG. 7B shows a comparison of durable CSP antibody responses between CPS immunized subjects and subjects experimentally infected with Plasmodium falciparum. Data points are shown for 1 day prior (C−1) and 35 (C+35), 140 (C+140), and 400 (C+400) days following exposure to the pathogen.

FIG. 7C shows a comparison of LSP-1 and CSP antibody responses between CPS immunized subjects and subjects experimentally infected with Plasmodium falciparum following re-challenge with the pathogen.

### DETAILED DESCRIPTION

The inventors have discovered numerous antigens and especially immunodominant antigens from the human pathogen Plasmodium falciparum by detecting and quantifying immune responses from individuals that have undergone a controlled and modified infection with Plasmodium falciparum sporozoites that was induced after prophylactic treatment with a suppressive drug (which produces a lasting immunity to Plasmodium falciparum).

Surprisingly, individuals that have undergone the natural course of the disease do not show significant immune response to a number of these antigens, and it is thus contemplated that the mechanism of immunity against malaria via natural course of infection and controlled and modified infection may follow substantially distinct pathways and use distinct antigens. It is generally contemplated that the herein presented antigens, portions and modifications thereof can be used by themselves, or more preferably, in combination with other antigens (typically immunodominant antigens) in the manufacture of therapeutic compositions and vaccines.

Among other suitable antigens, particularly contemplated antigens include liver stage antigen 1 (LSA-1), circumsporozoite protein (CSP), merozoite surface protein 2 (MSP2), and SET domain protein, and portions and modifications thereof, as well as any reasonable combination thereof.

More recently it had been shown that a regimen of a controlled and modified infection with sporozoites in individuals that were previously treated with a malaria suppressive drug (e.g., chloroquine) was successful in inducing protection against malaria from a blood meal of infected Anopheles in healthy individuals. While the sporozoites induced a strong immune response, the volunteers remained essentially asymptomatic. After a few months the so treated volunteers were subject to another infection and none became ill, showing that the immunity conferred by this treatment was highly effective. Based on this findings, the inventors set out to identify antigens associated with post-challenge immunity and contemplate that these antigens per se should be effective in eliciting immunity against Plasmodium falciparum without having to use sporozoite infection.

Accordingly, the present invention is directed to various antigens from the pathogenic organism Plasmodium falciparum, where the antigens have known reactivities to serum of a population of patients that have been treated with suppressive prophylaxis followed by sub-symptomatic infection (e.g., no significant paroxysm, headache, fever, shivering, arthralgia, vomiting, hemolytic anemia, jaundice, hemoglobinuria, and/or convulsions) and immunity. While not wishing to be bound by any theory or hypothesis, such controlled and modified infection is thought to allow the pathogen to multiply within host hepatocytes and to result in a mild blood stage infection that resolves quickly and produces a lasting immunity. Thus antigens from the organism that have a statistically high probability of eliciting a protective immune response are being presented to the host's immune system Immunogenic antigens, and especially highly immunogenic antigens identified from the immune response to such controlled and modified infection (i.e., post-challenge immunity associated antigens) will serve as the basis for an effective vaccine for the prevention of Plasmodium falciparum infection.

In one aspect of the inventive subject matter, an antigen composition comprises one, two, or more post-challenge immunity associated antigens, typically having quantified and known relative reactivities with respect to sera of a population infected with Plasmodium falciparum following prophylactic treatment. Most preferably, the antigens are LSA-1, CSP, MSP4, and/or SET domain protein, or fragments thereof. It is also contemplated that the antigens or fragments thereof are at least partially purified and/or recombinant. It is further contemplated that the known reactivities may be characterized by a variety of factors. However, it is particularly preferred that the known reactivities are characterized by strength of immunogenicity. Most typically, the post-challenge immunity associated antigens will be associated with a carrier that can be a pharmaceutically acceptable carrier for vaccination formulations, or a solid carrier for diagnostic use in which the post-challenge immunity associated antigens are individually addressable. For example, using compositions and methods according to the inventive subject matter, vaccine compositions can be formulated hat include a plurality of the post-challenge immunity associated antigens. Alternatively, contemplated compositions may be used to determine immunity status of a subject by first immunizing a subject with the post-challenge immunity associated antigens, and then measuring the strength and quality of the subject's immue response against the post-challenge immunity associated antigens, which will be indicative of the immunity against infection with Plasmodium falciparum.

In one exemplary manner of performing selected aspects of the inventive subject matter, it is preferred that at least part of the genome of Plasmodium falciparum is obtained and all potential open reading frames and splice mutations thereof are determined in silico. Once potential genes are identified, suitable primers are determined to provide amplicons of the entire Open Reading Frames (ORFs), or less preferably portions thereof, wherein the primers are preferably designed to allow facile subcloning into an expression system. Most preferably, the subcloning uses recombinase-based subcloning using unpurified PCR mixtures to avoid cloning bias, and the so obtained recombinant plasmids are polyclonally multiplied, which enables unbiased presentation of the amplicons. It is still further particularly preferred that the plasmid preparations are then subjected to an in vitro transcription/translation reaction to thereby provide the recombinant ORF peptide, which is then spotted or otherwise immobilized onto a suitable addressable carrier to produce an array. Such a carrier may be planar thereby forming a microarray of antigens in which the identity of each antigen is encoded by its position on the planar carrier. Planar carriers include but are not limited to coated and uncoated silica or glass surfaces (such as microscope slides), polymer surfaces, and membranes.

Alternatively, the carrier may be particulate and held in fluid suspension to form a fluid array. Such particulate carriers include but are not limited to silica or glass microspheres, silica or glass rods, and polymeric microspheres. Such particulate carriers may include an encoding system that identifies particulate carrier populations associated with specific antigens. Such encoding systems can include, but are not limited to dyes, fluorescent molecules, holographic labels, dimension of the particulate carrier, and particulate carrier shape. In yet another embodiment individual antigens may be used to coat individual wells in a microwell plate, producing an array of antigens in a familiar microwell plate format that is amenable to automated processing.

It should be recognized that the so prepared proteomes can then be exposed to serum of a population of control individuals and/or population of individuals that are known to have current or previous exposure to the above pathogen from which the ORFs were prepared. Antibodies present in serum that bind to one or more of the ORFs may then detected using known methods (e.g., secondary antibodies) known to those familiar with the art. In this manner, the entire proteome of the pathogen can be rapidly assessed for immunogenicity and potential binding with antibodies in serum.

Therefore, and among various other advantages, it should be especially recognized that contemplated compositions and methods presented herein will allow for preparation of vaccine compositions comprising a plurality of antigens with known affinity to target ORFs of Plasmodium falciparum that have been identified as reactive with sera from individuals that have active and effective immunity to this pathogen. As individual immune systems are known to exhibit significant variation with respect to antigen recognition, methods and compositions contemplated herein will allow statistically supported antigen identification to identify antigens, and especially immunodominant antigens in a population of patients that have received protective immunity to this pathogen. Consequently, multiple targets can be used to elicit an immune response, even where one or more of the targets may provide only a weak response. Exemplary suitable protocols for generation and screening of such proteome libraries are described in WO 2006/088492, WO 2008/140478, WO 2010/132054, WO 02/097051, U.S. Pat. App. Nos. 2004/0132132, 2006/224329, and 2003/082579, and U.S. Pat. No. 6,936,470, all of which are incorporated by reference herein.

With respect to the sequences identified herein, it should be further appreciated that the sequences need not be complete ORFs, but that suitable sequences may also be partial sequences (e.g., synthetic, recombinant, or isolated) that typically comprise at least part of an antigenic epitope. Thus, sequences contemplated herein may be identified as peptide sequence (or homologs thereof) or as DNA sequences encoding the antigenic peptide (partial or entire ORF). Similarly, chemically modified antigens, and/or orthologs of the polypeptides presented herein are also deemed suitable for use herein. Of course, it should be appreciated that all sequences contemplated herein may be modified to produce a homologous sequence. For example, where the sequence is a nucleic acid, contemplated modified sequences include those with non-natural nucleobases, insertions, deletions, transitions, and transversions. Therefore, all modified nucleic acid sequences that hybridize with contemplated sequences under stringent conditions are also included herein so long as the corresponding sequence is still immunoreactive. Furthermore, with respect to the reading frame for each of the sequences, it should be noted that the first base in the sequences is either the first base of the start codon or the first base in the first codon of the polypeptide that was identified with the methods and compositions provided herein. Most typically, the last three bases denote the stop codon, or the last base of the last codon of the polypeptide that was identified with the methods and compositions provided herein. Similarly, polypeptide sequences may be modified by post-translational modifications, including oxidation to form disulfied bonds, glycosylation, esterification, etc.

Most typically, post-challenge immunity associated antigens will be at least partially purified such that the antigen is present in an amount of at least 90%, more typically at least 99%, and most typically at least 99.9% purity. Thus, and viewed form a different perspective, it is generally preferred that the post-challenge immunity associated antigens are sufficienly pure to produce a single band upon Coomassie blue staining in a PAGE gel, and mopre preferably upon silver staining in a PAGE gel (where the gel is loaded with total protein not to exceed recommended marker protein load for the particular gel/pocket size). Among other suitable manners of purification, affinity purification methods are especially preferred, however, other purification methods are also deemed suitable and a well known to a person of ordinary skill in the art.

In still further contemplated aspects, antigens associated with immunity are identified by selecting for an antigen by comparison of at least two series of tests, wherein one series of tests is typically the sub-population (e g, immunized, or challenged under prophylaxix with suppressive drugs) and the other series of tests is the control group (e.g. non-immunized) both before and after experimental infection. Still further, it is generally preferred that the series of tests also include a negative control against which the potential immunodominant antigens are compared. Such an immunized sub-population may be produced by treating individuals with an agent that permits a subsequently introduced pathogen to multiply, thereby inducing an immune response, while modifying the course of the infection. It should be appreciated that compositions comprising one or preferably more selected immunodominant antigens can be prepared that will have a statistically high probability to elicit or have elicited a protective immune response. Moreover, as the antigens presented herein are immunodominant antigens, it should be noted that vaccine compositions can be prepared with known or predictable immunogenicity.

Alternatively, antigens may also be identified from a patient population that has been infected with stage-arrested forms (e.g., one or more mutant forms of sporozoits that are developmentally arrested to remain in liver stage and not to transfer to erythrocytes), or that have been infected with replication deficient forms. It is therefore also contemplated that the compositions and methods presented herein may be used to generate stage specific immunity to a person (i e, immunity with immune reaction to only one stage of the parasite life cycle, for example, liver stage, pre-erythrocyte stage, erythrocyte stage, etc.).

Most typically, where contemplated post-challenge immunity associated antigens are used in a vaccine formulation, it is contemplated that individuals vaccinated with the post-challenge immunity associated antigens will have durable and protective immunity against Plasmodium falciparum infection that is effective to suppress growth, propagation, and development of Plasmodium falciparum and thus protects from symptomatic disease. Thus, immunity is expected to last at least 12 months, more typically at least 24 months for at least 70%, more typically at leasy 80%, and most typically at least 90% of all individuals subjected to vaccination with post-challenge immunity associated antigens.

Among other antigens, the following antigens of Plasmodium falciparum have been particularly associated with effective and enduring immunity: Liver stage antigen 1 (LSA-1), circumsporozoite protein (CSP), merozoite surface protein 2 (MSP2), and SET domain protein. Therefore, any combination of two, three, or all of LSA-1, CSP, MSP2, and SET domain protein, post-translational modifications, and fragments thereof may be components of a vaccine preparation for providing effective and enduring immunity to Plasmodium falciparum. For example, suitable combinations include LSA-1, CSP, and MSP4, or fragments thereof, LSA-1, CSP, and SET domain protein, or fragments thereof, LSA-1, MSP4, and SET domain protein, or fragments thereof, and/or CSP, MSP4, and SET domain protein, or fragments thereof. Moreover, such combinations may further include additional antigens of Plasmodium falciparum known to be antigenic or effective as eliciting an immune response.

Recognizing that durable immunity (protective immunity greater than 2 years) to Plasmodium falciparum is associated with a strong and persistent immune response to specific antigens, another aspect of the invention is a method for determining a potentially susceptible individual's immune status relative to Plasmodium falciparum. In this embodiment one or more post-challenge immunity associated antigens form the basis of an assay for immune competency to this parasite. An immune response of a potentially susceptible individual to said antigen or antigens could be characterized by methods known to those in the art, including immunoassays. Devices that incorporate such assays could include lateral flow or “dipstick” devices suitable for point of care testing in the field, microwell plate based enzyme linked immunosorbent assays suitable for screening on automated systems, and separation free immunoassays suitable for use on clinical testing systems. In this aspect of the invention a strong immune response to such antigens would indicate resistance to P. falciparum infection, whereas a reduced or absent immune response to such antigens would indicate susceptibility to P. falciparum infection. In one embodiment these antigens may be selected from the group comprising LSA-1, CSP, MSP4, and SET domain protein, or fragments thereof.

In these examples, each of the antigens was characterized, inter alia, with regard to individual and relative reactivities with sera taken from individuals immunized to Plasmodium falciparum by chloroquine prophylaxis followed by experimental infection with sporozoites (CPS immunization), and comparison to results from individuals that had experienced infection without prior treatment. Most typically, reactivity was measured as strength of immunogenicity (e.g., such that average binding affinity and/or average quantity of the antibodies produced a significant signal intensity). In still further contemplated aspects, the antigens presented herein may be employed in the manufacture of a vaccine that comprises at least one, and more typically at least two of the (preferably immunodominant) antigens. More preferably, however, contemplated vaccines will include between two and six, and even more antigens, of which at least one of the antigens is an immunodominant antigen.

With respect to suitable formulations of vaccines, it should be recognized that all known manners of producing such vaccines are deemed appropriate for use herein, and a person of ordinary skill in the art will be readily able to produce such vaccines without undue experimentation (see e.g., “Vaccine Adjuvants and Delivery Systems” by Manmohan Singh; Wiley-Interscience (Jun. 29, 2007), ISBN: 0471739073; or “Vaccine Protocols” (Methods in Molecular Medicine) by Andrew Robinson, Martin P. Cranage, and Michael J. Hudson; Humana Press; 2 edition (Aug. 27, 2003); ISBN: 1588291405). Therefore, suitable vaccines may be formulated as injectable solutions, or suspensions, intranasal formulations, or as oral formulations.

### EXAMPLES

### Test Samples

An open-label clinical trial was done at the Radboud University Nijmegen Medical Centre (Nijmegen, Netherlands), from November to December, 2009, 28 months after the start of the previous challenge infection. Ten newly recruited malaria-naive volunteers aged 18 to 35 years were screened for eligibility for inclusion in the control group based on medical and family history, physical examination, and general haematological and biochemical screening including HIV, hepatitis B, and hepatitis C serology, urine toxicology screening, and a pregnancy test. The main exclusion criteria were residence in a malaria-endemic region within the previous 6 months, positive P. falciparum serology, or an estimated 10-year risk greater than 5% of developing a cardiac event as estimated by the systematic coronary evaluation system. All volunteers gave written informed consent before inclusion. The trial was done in accordance with good clinical practice and approved by the Central Committee for Research Involving Human Subjects of The Netherlands (CCMO NL24193.091.09).

### Protein Microarray Chip Fabrication and Probing Methods

All ORFs from P. falciparum 3D7 strain genomic DNA were amplified and cloned using a high-throughput PCR and recombination cloning method and microarrays were fabricated and probed as described by Liang, L., et al (PLoS Negl Trop Dis. 2010 May; 4(5): e673). Plasmids were expressed at 24° C. for 16 hrs in in vitro transcription/translation E. coli reactions (Expressway Maxi kits from Invitrogen in Carlsbad, Calif.), according to the manufacturer's instructions. For microarrays, 10 μl of reaction was mixed with 3.3 μl 0.2% Tween 20 to give a final concentration of 0.05% Tween 20, and printed onto nitrocellulose coated glass FAST slides (Whatman in Piscataway, N.J.) using an Omni Grid 100 microarray printer (Genomic Solutions in Boston, Mass.). Human sera samples were diluted to 1:200 with 10 mg/ml E. coli lysate (Mclab in San Francisco, Calif.). Microarray slides were incubated in biotin-conjugated secondary antibody (Jackson ImmunoResearch in West Grove, Pa.) diluted 1/200 in blocking buffer, and detected by incubation with streptavidin-conjugated SureLight® P-3 (Columbia Biosciences in Columbia, Md.). The slides were washed and air dried by brief centrifugation. Microarray slides were scanned and analyzed using a Perkin Elmer ScanArray Express HT microarray scanner. (Perkin Elmer in Waltham, Mass.). Intensities were quantified using QuantArray software (Packard BioChip Technologies in Billerica, Mass.). All signal intensities were corrected for spot-specific background.

### Data Analysis

Microarray spot intensities were quantified using QuantArray software utilizing automatic background subtraction for each spot. Proteins were considered to be seroreactive if signal intensity was greater than the average signal intensity of the reaction without plasmid, plus 2.5-times the standard deviation. “No DNA” controls consisting of reactions without addition of plasmid were averaged and used to subtract background reactivity from the unmanipulated raw data. Results herein are expressed as signal intensity. To normalize differences in background reactivity seen among the different individuals, pre-immune background reactivity for each antigen was subtracted from the data in subsequent time points for each individual. Differentially reactive proteins between groups were determined using a Bayes regularized t-test; a p-value smaller than 0.05 was considered significant.

### Proteome Array Design and Construction

The protein microarray used for this study contains 853 proteins that were selected based on their serum reactivity in a series of worldwide cohorts. An array containing 4,300 proteins was probed with specimens from individuals with naturally acquired immunity to malaria living in Mali, Kenya, Ghana, and Angola. This 4,300 element array was also probed with sera from subjects in an irradiated sporozoite vaccine clinical trial which included individuals that were protected and unprotected after vaccination, as well as unimmunized positive infectivity controls. Analysis of the results of these studies produced a list of the most reactive 853 antigens, and these were printed on the array used for this work.

### Specimen Collection

The specimens used for this work were derived from a clinical study evaluating protective efficacy of chloroquine prophylaxis followed by sporozoite infection (CPS immunization) against an experimental malaria sporozoite challenge. Fifteen healthy volunteers in the study (10 assigned to a vaccine group and 5 assigned to a control group) were exposed to bites of mosquitoes once a month for 3 months while receiving a prophylactic regimen of chloroquine. The vaccine group was exposed to mosquitoes that were infected with Plasmodium falciparum, and the control group was exposed to mosquitoes that were not infected with the malaria parasite. One month after the discontinuation of chloroquine treatment, protection was assessed by homologous challenge using mosquitoes infected with P. falciparum. All 5 individuals in the control group experienced clinical malaria disease and patent parasitemia after challenge; all 10 individuals in the immunized group were protected from disease. More than 2 years (874 days) after challenge, 6 of the 10 immunized individuals and 5 additional volunteers were subjected to challenge with bites of mosquitoes that were infected with the malaria parasite. All 5 individuals that were not previously immunized and two previously immunized individuals experienced clinical malaria disease and patent parasitemia after this re-challenge. Plasma samples from each individual in the study were taken 1 day prior to immunization, 1 day prior to challenge, 35 days, 140 days and 400 days post challenge, 1 day prior to re-challenge, and 35 days post re-challenge. Each serum sample from each individual was analyzed for the presence of IgG antibodies on the protein microarray.

### Human Antibody Profiles

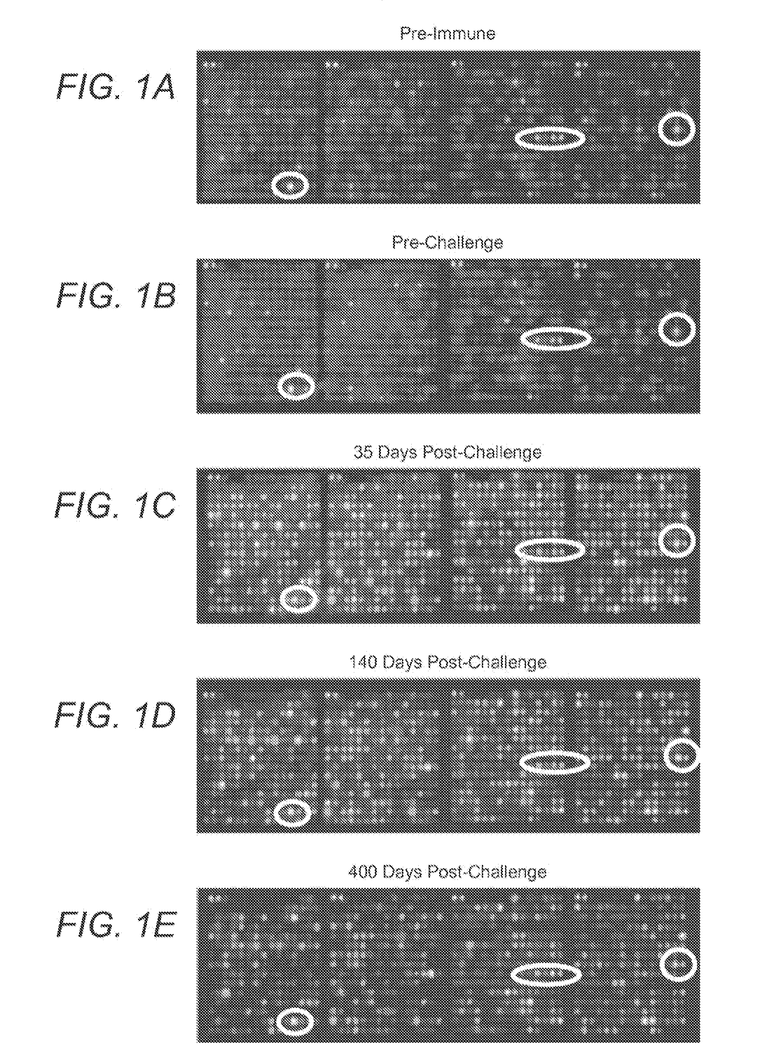

The images in FIG. 1 show arrays probed with the series of time course specimens from one individual in the study from the control group who was not immunized prior to challenge and developed malaria disease after challenge. Pre-immune (FIG. 1A) and pre-challenge (FIG. 1B) arrays showed similar low background reactivity, indicating no evidence of cross reactivity against malaria antigens from antibodies produced against mosquito salivary proteins. Several strong background reactive antigens (circled) show a similar level of reactivity throughout the entire time course and were used as internal positive reference signals. Arrays probed with serum from 35 days post challenge (FIG. 1C) showed increased reactivity against hundreds of malaria antigens, with levels of reactivity declining 140 days (FIG. 1D) and 400 days (FIG. 1E) post challenge.

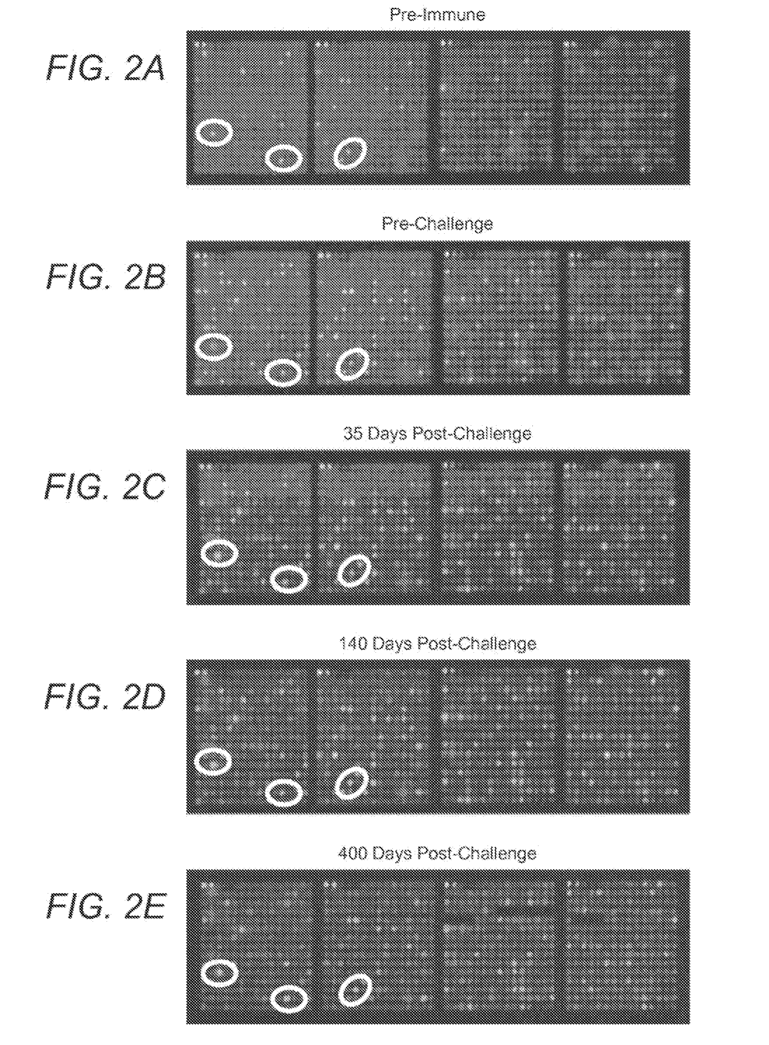

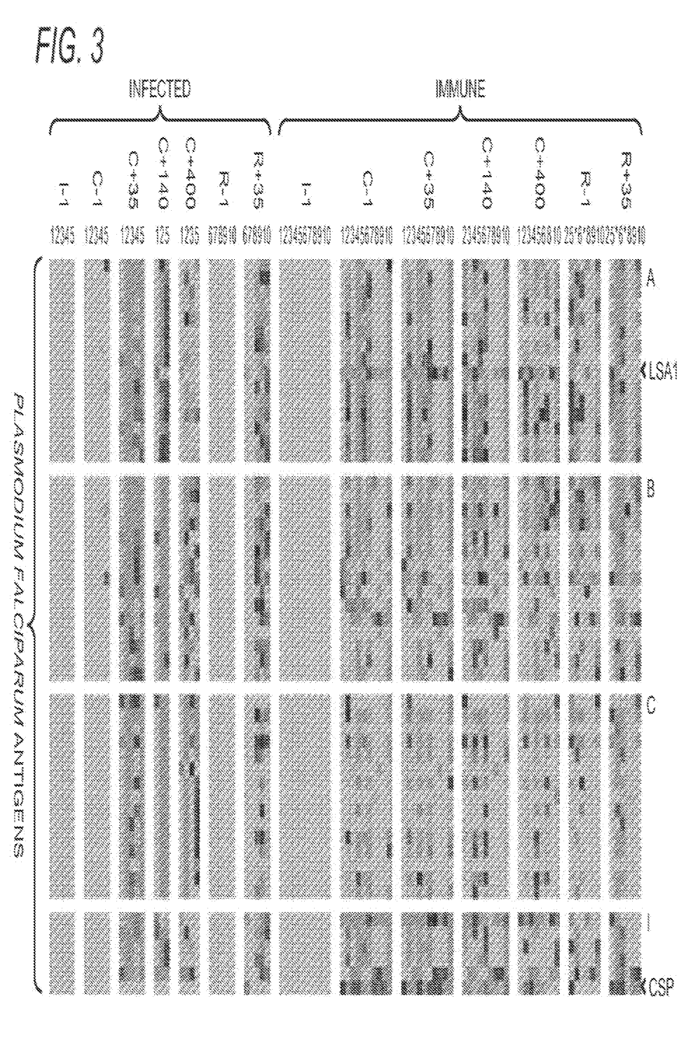

Longitudinal samples from a CPS immunized individual are shown in FIG. 2. An increased antibody response due to the immunization is seen in the pre-challenge sample (FIGS. 2A and 2B). There is no apparent change in this antibody profile after challenge (FIG. 2C), indicating that the infection was cleared at an early step in the parasite development cycle with no new antigens significantly exposed to the immune system as a result of the challenge. The antibody response against most of the malaria antigens gradually declined 140 (FIG. 2D) and 400 days (FIG. 2E) post challenge. The reference background reactivity spots (circled) did not change throughout this time course.

To normalize differences in background reactivity observed between individuals, pre-immune background reactivity for each antigen from each individual was subtracted from the data in subsequent time points and a color coded “heatmap” of the normalized data generated. An exemplary heatmap is shown in FIG. 3. The antigens are in rows and the human samples are in columns. The antigens in groups A, B, and C are identified in control individuals and antigens in Group I are identified in immunized group. The identity of each antigen has been listed in Table 1.

TABLE 1

| | Antigen ID | Gene ID | Product Description |
|---|---|---|---|
| A | PFI0580ce2s2 | PFI0580c | falstatin |
| | PFF0765ce2s3 | PFF0765c | conserved Plasmodium protein, unknown function |
| | PFE1600we2s1 | PFE1600w | Plasmodium exported protein (PHISTb), unknown function |
| | PF07_0053e1s4 | PF07_0053 | conserved Plasmodium protein, unknown function |
| | PFD0225w-s5 | PFD0225w | conserved Plasmodium membrane protein, unknown function |
| | PF11_0404e2s3 | PF11_0404 | transcription factor with AP2 domain(s), putative |
| | PF14_0512e1s1 | PF14_0512 | conserved Plasmodium protein, unknown function |
| | PF13_0210 | PF13_0210 | conserved Plasmodium protein, unknown function |
| | PF10_0356e1s2 | PF10_0356 | liver stage antigen 1 |
| | PFC0270we5s5 | PFC0270w | activator of Hsp90 ATPase, putative |
| | PF10_0138-s2 | PF10_0138 | conserved Plasmodium protein, unknown function |
| | PFA0410w-s1 | PFA0410w | conserved Plasmodium protein, unknown function |
| | MAL7P1.32 | MAL7P1.32 | nucleotide excision repair protein, putative |
| | PF14_0649e2s1 | PF14_0649 | conserved Plasmodium protein, unknown function |
| | PFF1485we2s2 | PFF1485w | conserved Plasmodium protein, unknown function |
| B | MAL7P1.14e1s1 | MAL7P1.14 | conserved Plasmodium protein, unknown function |
| | PF11_0074e2s6 | PF11_0074 | exonuclease, putative |
| | PF07_0026e1s1 | PF07_0026 | ubiquitin-protein ligase e3, putative |
| | PF08_0024e1s1 | PF08_0024 | conserved Plasmodium protein, unknown function |
| | PFF0995ce1s1 | PFF0995c | merozoite surface protein 10 |
| | PFC0425we1s3 | PFC0425w | conserved Plasmodium protein, unknown function |
| | PFE1590w | PFE1590w | early transcribed membrane protein 5 |
| | PFE0120ce1s1 | PFE0120c | merozoite surface protein 8, ring-stage membrane protein 1 |
| | PF10_0025e2s2 | PF10_0025 | PF70 protein |
| | PFD1015w | PFD1015w | hypothetical protein, conserved |
| | PFB0300c | PFB0300c | merozoite surface protein 2 |
| | PFB0915we2s2 | PFB0915w | liver stage antigen 3 |
| | PF11_0107e1s3 | PF11_0107 | conserved Plasmodium protein, unknown function |
| | PF14_0188e1s2 | PF14_0188 | conserved Plasmodium membrane protein, unknown function |
| | PFL0075we1s1 | PFL0075w | XPA binding protein 1, putative |
| C | PF14_0016e1s1 | PF14_0016 | early transcribed membrane protein 14.1 |
| | PF14_0344e1s1 | PF14_0344 | translocon component PTEX150 |
| | PF10_0281e1s1 | PF10_0281 | merozoite TRAP-like protein |
| | PFI0320we1s1 | PFI0320w | arginase, putative |
| | PFL0440ce1s1 | PFL0440c | zinc finger protein, putative |
| | PF14_0102e1s1 | PF14_0102 | rhoptry-associated protein 1 |
| | PF14_0274e1s1 | PF14_0274 | diphthamide synthesis protein, putative |
| | PFC0120we1s1 | PFC0120w | cytoadherence linked asexual protein 3.1 |
| | PF11_0270e1s1 | PF11_0270 | threonine - tRNA ligase, putative |
| | PF13_0208e1s2 | PF13_0208 | exoribonuclease, putative |
| | MAL8P1.23-s9 | MAL8P1.23 | ubiquitin-protein ligase 1, putative |
| | PF11_0240e2s5 | PF11_0240 | dynein heavy chain, putative |
| | PFB0775we1s1 | PFB0775w | conserved Plasmodium protein, unknown function |
| | PFA0295ce1s1 | PFA0295c | conserved Plasmodium protein, unknown function |
| | PFL2390ce1s3 | PFL2390c | conserved Plasmodium protein, unknown function |
| I | PF10_0356e1s2 | PF10_0356 | liver stage antigen 1 |
| | PFF0995ce1s1 | PFF0995c | merozoite surface protein 10 |
| | PF07_0053e1s4 | PF07_0053 | conserved Plasmodium protein, unknown function |
| | PF11_0404e2s3 | PF11_0404 | transcription factor with AP2 domain(s), putative |
| | PFB0300c | PFB0300c | merozoite surface protein 2 |
| | PFC0210c | PFC0210c | circumsporozoite (CS) protein |

In the control group I−1 and C−1 are low, indicating no change in background reactivity resulting from the bites of uninfected mosquitos. At 35 days post challenge (C+35) reactivity is high except for one individual who did not react strongly to the challenge. At the later time points the reactivity declines. For the re-challenge phase, new control subjects were enrolled, the R−1 data are at baseline and the R+35 values are increased as expected.

In CPS immunized subjects background pre-immune reactivity was also subtracted from each subsequent time point. Elevated reactivity can be seen after completion of the immunization phase one day before challenge (C−1), and reactivity declines gradually at the later time points. Surprisingly, it was found that reactivity above baseline persists two years later at the R−1 time point and that some of these individuals successfully completed a second experimental challenge. In both the control and CPS immunized groups there was variation in the response between individuals. One of the control individuals had a weak response to the challenge but the remaining 4 responded strongly. The response to CPS immunization varied between the individuals in the study, except for LSA-1 and CS Protein in which a strong response was noted in all individuals until at least 140 days post challenge.

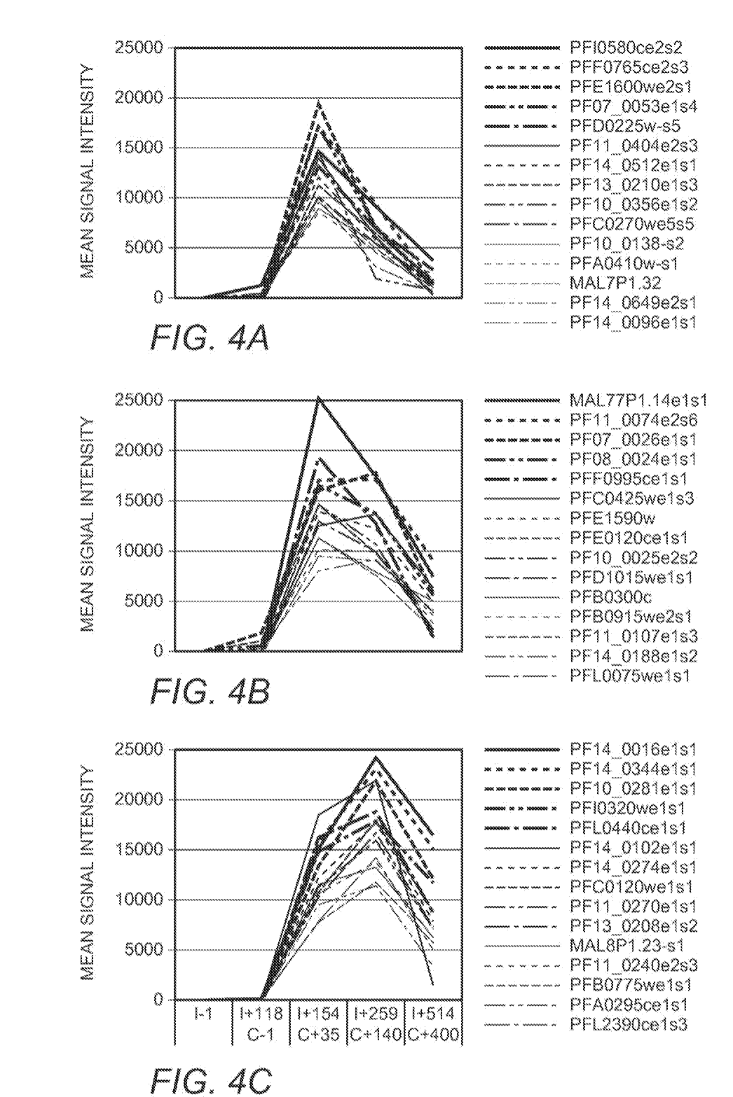

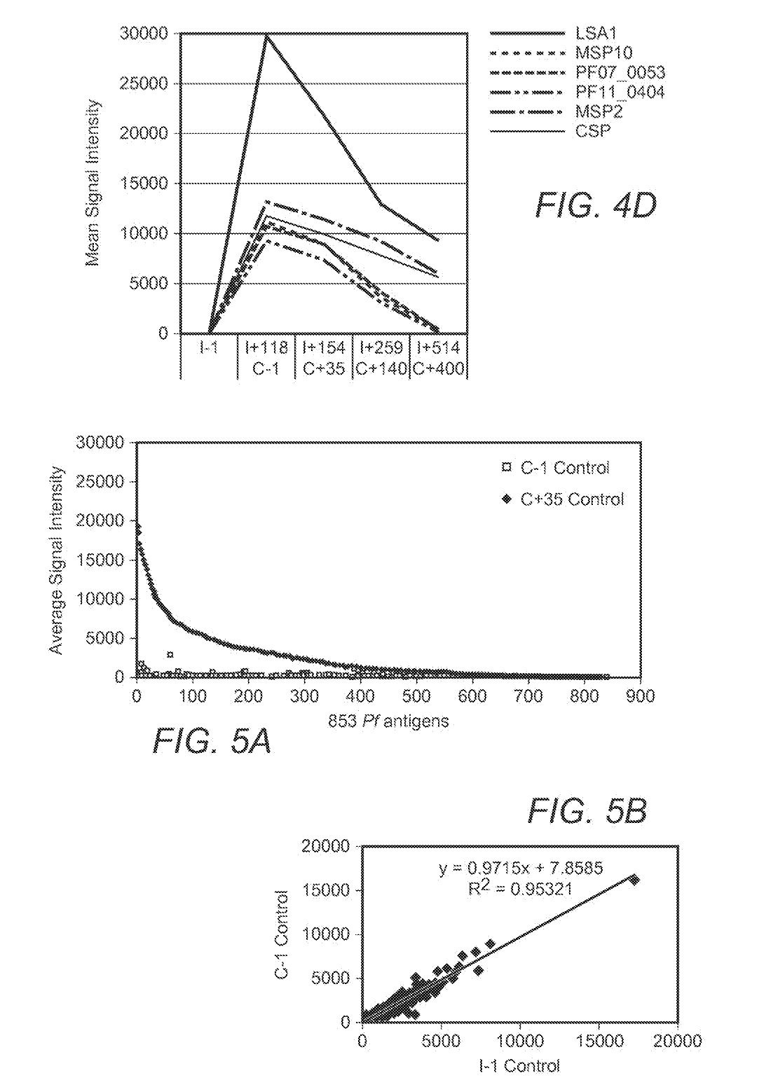

Mean antibody reactivity from the five control individuals to specific Plasmodium falciparum proteins are plotted in FIG. 4 as a function of time post challenge showing three distinct kinetic patterns. There are shown 15 antigens to which antibody levels peak 35 days post challenge and sharply decline thereafter (FIG. 4A), 15 antibodies peak 35 days post challenge but decline more slowly (FIG. 4B), and 15 antibody responses peak at a later time post-challenge (FIG. 4C). The reasons for these different kinetic patterns may be related to the stage of the organism in which they are expressed, their physical or functional properties, subcellular localization, or their absolute level and duration of expression.

Overall the mean peak reactivity of antigens in the immunized group was lower than in the control group (FIG. 4D). LSA-1 was the most reactive antigen observed in the study; CSP was another notable antigen induced by CPS immunization.

### Characterization of Antibody Profiles Associated with CPS Immunization and Malaria Disease

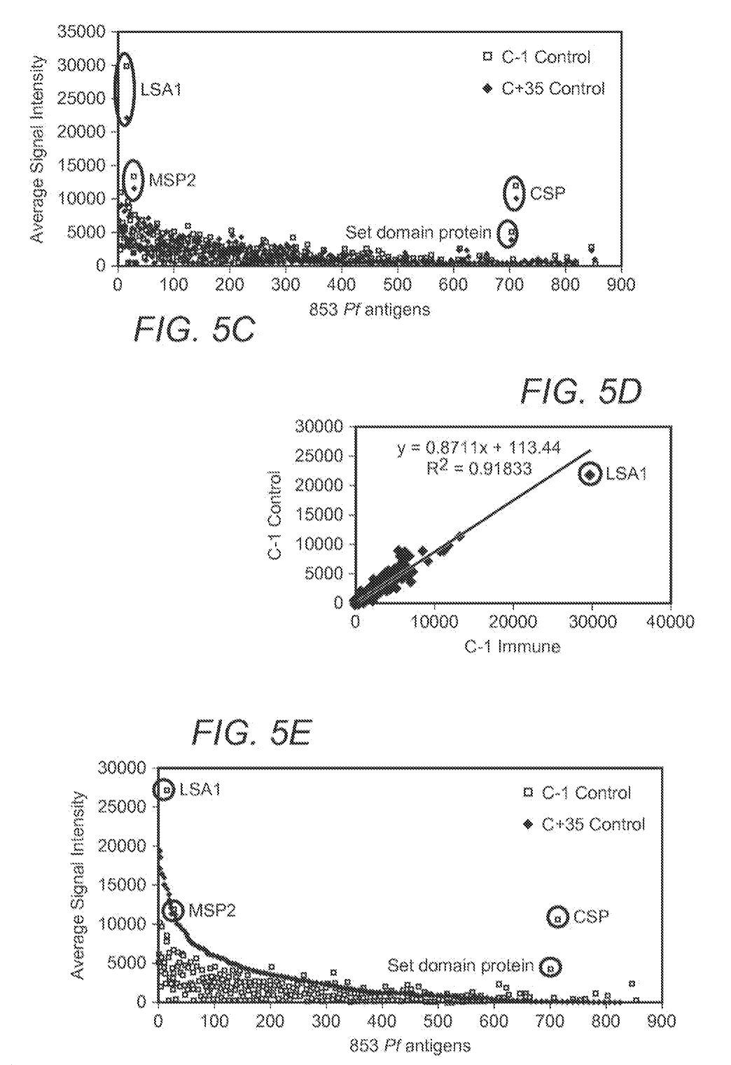

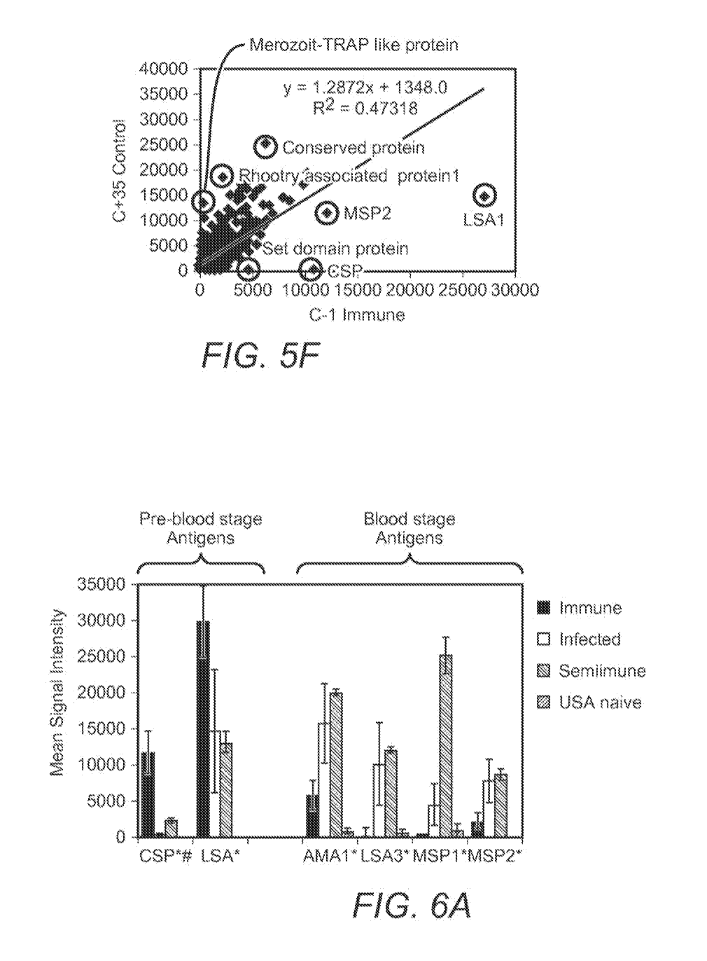

The scatterplot in FIG. 5B compares the average reactivity of the five control subjects for each antigen in pre-immune sera (I−1) with the one day pre-challenge sera (C−1) showing a good correlation and confirming that the data collected from these two time points do not differ significantly from one another (R²=0.95 and slope=0.97). This result confirms no evidence of cross reactivity resulting from the multiple exposures to saliva proteins from uninfected mosquitos. In FIG. 5A there are 853 antigens plotted along the X-axis and the average array signal intensity for each antigen after pre-immune subtraction is plotted on the Y-axis. As expected, the average reactivity 1 day prior to challenge (C−1) produces data that is near the baseline. The average pre-immune corrected data 35 days post challenge (C+35) show a strong antibody response against hundreds of P. falciparum antigens in experimentally infected individuals who develop patent blood stage parasitemia.

FIG. 5C plots the average reactivity of the 10 individuals in the CPS immunized group comparing the reactivity 1 day before challenge and 35 days post challenge. The antigens are plotted in the same order as FIG. 5A. CPS immunized individuals also react against hundreds of P. falciparum antigens, but the overall antibody response is lower than from the infected controls. In particular, immunized subjects react against LSA1, CSP, MSP2, Set Doman Protein, and MSP4. There is no significant difference between the C−1 and C+35 signals in the Immune Group and these protected individuals do not experience a significant change in immune status from the live organism challenge. The scatterplot comparing pre- and post-challenge shows a nearly linear correlation (R²=0.94 and slope=0.93), confirming this conclusion (FIG. 5D). This result indicates that CPS immunized individuals did not develop blood parasites after challenge and consequently no new antigens were exposed to the immune system.

FIG. 5E compares the antibody response between immunized individuals 1 day before challenge and naïve individuals 35 days post challenge. Parasistemic individuals exhibit higher antibody titers against most of the antigens on the array. Notable exceptions include LSA1, CSP, sporozoite SET Domain protein, and MSP2, to which antibody responses are surprisingly higher in immunized individuals than in parasitemic individuals. A scatterplot of this data (FIG. 5F) confirms that the antibody response between immunized individuals and infected parasitemic subjects is different both in terms of intensity (slope=1.3) and the antibody profile (R²=0.47). Antibodies against merozoite TRAP like protein, rhoptry associated protein 1 and a conserved Plasmodium protein are higher in parasitemic individuals than in immunized individuals. A list of 5 most reactive antigens from each group is shown in Table 2.

TABLE 2

| Top 5 Ags in C+35 Control | | Top 5 Ags in C-1 Immune | |
|---|---|---|---|
| Ag ID | Product Description | Ag ID | Product Description |
| MAL7P1.14e1s1 | conserved Plasmodium membrane protein, unknown function | PF10_0356e1s2 | liver stage antigen 1 |
| PFE1600we2s1 | Plasmodium exported protein (PHISTb) | PFB0300c | merozoite surface protein 2 |
| PFF0995ce1s1 | merozoite surface protein 10 | PFC0210c | circumsporozoite (CS) protein |
| PF14_0102e1s1 | rhoptry-associated protein 1 | PFF0995ce1s1 | merozoite surface protein 10 |
| PFF0765ce2s3 | conserved Plasmodium protein, unknown function | PF07_0053e1s4 | conserved Plasmodium protein, unknown function |

### Differential Reactivity of Preerythrocytic and Blood Stage Antigens

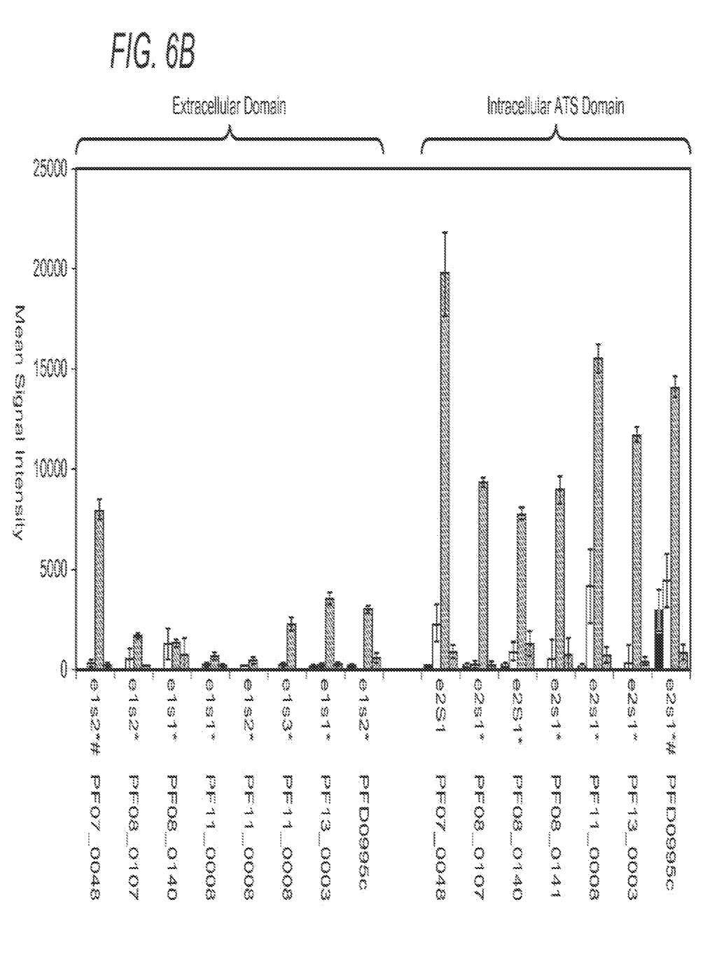

Since chloroquine blocks parasite development at the erythrocyte stage, ‘CPS Immunization’ may induce immune responses against pre-erythrocytic antigens and not against antigens expressed exclusively in blood stage parasites. The analyses in FIG. 4 provide an empirical framework that supports this by comparing antibody reactivity of pre-erythrocytic and blood stage antigens in individuals either ‘CPS Immunized’, experimentally infected, or naturally immune adults (semi-immune) residing in an endemic environment (Tanzania). Surprisingly, there are two pre-erythrocytic antigens, Circumsporozoite Protein (CS Protein) and Liver Stage Antigen 1 (LSA-1) that are significantly more reactive in CPS immunized individuals than in either experimentally infected or semi-immune subjects (FIG. 6A). LSA1 reactivity in CPS immunized individuals was the most reactive antigen noted. AMA1, MSP1, MSP2, and LSA3 are seen by mass spectrometry in blood stage parasites, and experimentally infected and semi-immune individuals react more strongly to these antigens than CPS immunized subjects. FIG. 6B shows data from another characteristic blood stage antigen expressed in erythrocytes, PfEMP1, which generates high antibody reactivity in naturally exposed semi-immune individuals and a detectable response in non-immunized infected controls, but not in CPS immunized subjects.

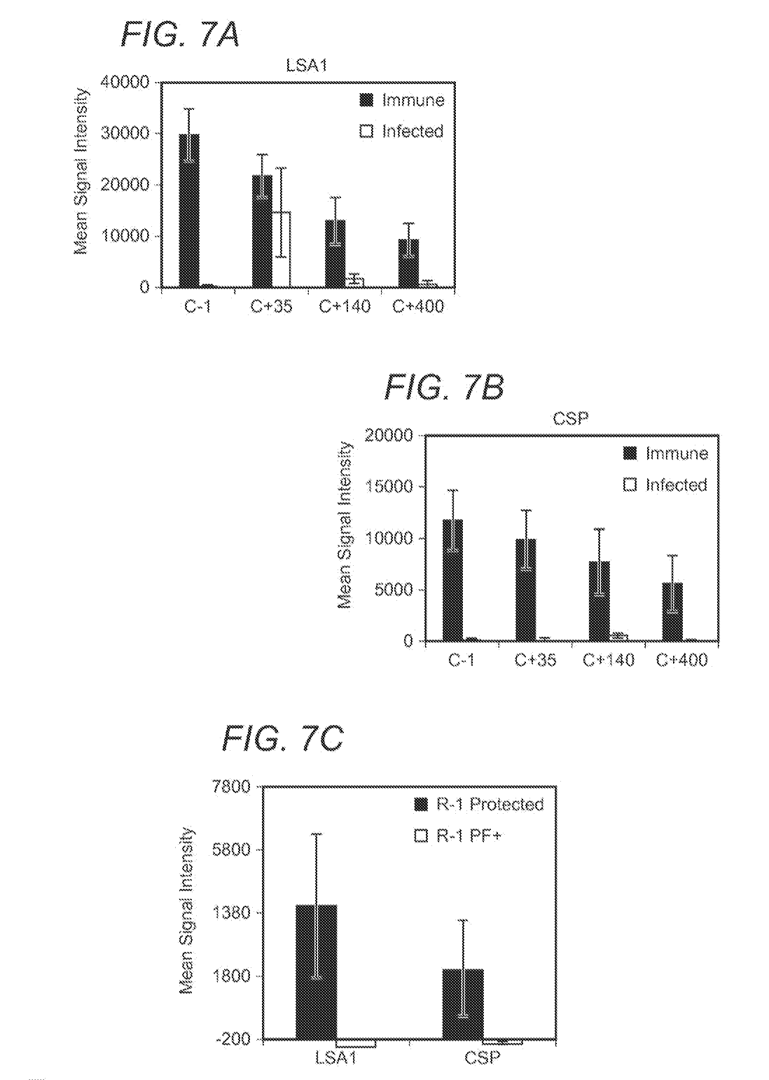

The results illustrated in FIGS. 7A and 7B highlight the differences in the level and kinetics of the antibody responses between CPS immunized individuals and experimentally infected subjects. Infection induces a transient response against LSA-1, but CPS immunization induces a durable response to this antigen lasting more than a year. CPS immunization also induces a strong and durable response to the CS Protein, whereas the response in experimentally infected individuals to CS Protein is negligible.

Six individuals from the immunized group were re-challenged with viable sporozoites two years later, and four retained durable immunity while two became susceptible and developed malaria. The results plotted in FIG. 7C show that the protected individuals from this study retained significant antibody to LSA-1 and CS Protein two years after immunization, whereas the two susceptible individuals (PF+) had insignificant reactivity against these two antigens. This strong and surprising association between the protective activity of CPS Immunization and the durable antibody responses to CS Protein and LSA1 indicates that these two proteins may be potential vaccine antigen candidates.

Thus, specific embodiments and applications of antigenic compositions and methods for Plasmodium falciparum have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

1. An antigenic composition comprising:
a plurality of at least partially purified post-challenge immunity associated antigens of Plasmodium falciparum;
wherein the plurality of post-challenge immunity associated antigens comprise at least three antigens selected from the group consisting of: an LSA-1 or an immunogenic fragment thereof, a CSP or an immunogenic fragment thereof, a MSP4 or an immunogenic fragment thereof, and a SET domain protein or an immunogenic fragment thereof; and
a carrier associated with the plurality of post-challenge immunity associated antigens.

2. The antigenic composition of claim 1, wherein the at least three antigens are: an LSA-1 or an immunogenic fragment thereof, a CSP or an immunogenic fragment thereof, and a MSP4 or an immunogenic fragment thereof.

3. The antigenic composition of claim 1, wherein the at least three antigens are: an LSA-1 or an immunogenic fragment thereof, a CSP or an immunogenic fragment thereof, and a SET domain protein or an immunogenic fragment thereof.

4. The antigenic composition of claim 1, wherein the at least three antigens are: an LSA-1 or an immunogenic fragment thereof, an MSP4 or an immunogenic fragment thereof, and a SET domain protein or an immunogenic fragment thereof.

5. The antigenic composition of claim 1, wherein the at least three antigens are: a CSP or an immunogenic fragment thereof, an MSP4 or an immunogenic fragment thereof, and a SET domain protein or an immunogenic fragment thereof.

6. The antigenic composition of claim 1, wherein the plurality of post-challenge immunity associated antigens comprises a LSA-1 or an immunogenic fragment thereof, a CSP or an immunogenic fragment thereof, a MSP4 or an immunogenic fragment thereof, and a SET domain protein or an immunogenic fragment thereof.

7. The antigenic composition of claim 1, wherein the carrier comprises a pharmaceutically acceptable carrier, and the composition is formulated as a vaccine formulation.

8. The antigenic composition of claim 1, wherein the carrier comprises a solid phase to which the plurality of post-challenge immunity associated antigens are coupled in an individually addressable manner.

9. The antigenic composition of claim 8 wherein the carrier is part of a disposable diagnostic test device.

10. A method of developing a multivalent vaccine formulation that confers persistent immunity against Plasmodium falciparum, comprising:
identifying a plurality of post-challenge immunity associated antigens of Plasmodium falciparum;
at least partially purifying each of the post-challenge immunity associated antigens; and
including the plurality of at least partially purified post-challenge immunity associated antigens into a vaccine formulation comprising a pharmaceutically acceptable carrier and optionally an adjuvant.

11. The method of claim 10, wherein the wherein the plurality of antigens comprises at least one antigen selected from the group consisting of: an LSA-1 or an immunogenic fragment thereof, a CSP or an immunogenic fragment thereof, a MSP4 or an immunogenic fragment thereof, and a SET domain protein or an immunogenic fragment thereof.

12. The method of claim 10 wherein the wherein the plurality of antigens comprises at least two antigens selected from the group consisting of: an LSA-1 or an immunogenic fragment thereof, a CSP or an immunogenic fragment thereof, a MSP4 or an immunogenic fragment thereof, and a SET domain protein or an immunogenic fragment thereof.

13. The method of claim 10, wherein the wherein the plurality of antigens comprises at least three antigens selected from the group consisting of: an LSA-1 or an immunogenic fragment thereof, a CSP or an immunogenic fragment thereof, a MSP4 or an immunogenic fragment thereof, and a SET domain protein or an immunogenic fragment thereof.

14. The method of claim 10, wherein the step of identifying comprises administration of a suppressive drug to a mammal prior to a step of infecting the mammal with a dose of sporozoites of Plasmodium falciparum, wherein the dose is effective to confer immunity to Plasmodium falciparum to the mammal without development of symptomatic disease of Plasmodium falciparum in the mammal.

15. A method for assessing the immune competence of an individual to a Plasmodium falciparum, comprising:
administering a plurality of post-challenge immunity associated antigens of the Plasmodium falciparum to the individual;
obtaining a blood sample from the individual;
determining and Quantifying the amount of of antibodies against each of the plurality of post-challenge immunity associated antigens in the blood sample, and comparing the determined quantities of antibodies against a respective threshold value of antibodies against the same plurality of post-challenge immunity associated antigens;
wherein the respective threshold value of antibodies is based on a plurality of individuals that have persistent and effective immunity against the Plasmodium falciparum, and the quantities of antibodies above the respective threshold value is indicative of immunity to the Plasmodium falciparum.

16. The method of claim 15, wherein the wherein the plurality of post-challenge immunity associated antigens includes at least one antigen selected from the group consisting of: an LSA-1 or an immunogenic fragment thereof, a CSP or an immunogenic fragment thereof, a MSP4 or an immunogenic fragment thereof, and a SET domain protein or an immunogenic fragment thereof.

17. The method of claim 15, wherein the wherein the plurality of post-challenge immunity associated antigens includes at least two antigens selected from the group consisting of: an LSA-1 or an immunogenic fragment thereof, a CSP or an immunogenic fragment thereof, a MSP4 or an immunogenic fragment thereof, and a SET domain protein or an immunogenic fragment thereof.

18. The method of claim 15 wherein the wherein the plurality of post-challenge immunity associated antigens includes at least three antigens selected from the group consisting of: an LSA-1 or an immunogenic fragment thereof, a CSP or an immunogenic fragment thereof, a MSP4 or an immunogenic fragment thereof, and a SET domain protein or an immunogenic fragment thereof.

19. The method of claim 15, wherein the individual is naive to infection with Plasmodium falciparum.

20. (canceled)

21. The method of claim 15, wherein the individual is a human.
